(19) United States
(12) Patent Application Publication
(10) Pub. No.: US 2011/0224673 A1
(43) Pub. Date: Sep. 15, 2011

(54) JOINT IMPLANT AND PROSTHESIS AND METHOD

(75) Inventor: Matthew J. Smith, Columbia, MO (US)

(73) Assignee: THE CURATORS OF THE UNIVERSITY OF MISSOURI, Columbia, MO (US)

(21) Appl. No.: 13/044,740

(22) Filed: Mar. 10, 2011

Related U.S. Application Data
(60) Provisional application No. 61/340,085, filed on Mar. 11, 2010.

(51) Int. Cl. A61B 17/56; A61F 2/30

(52) U.S. Cl. 606/87; 623/23.39

(57) ABSTRACT

Disclosed herein is a minimally invasive ball joint implant and prosthesis for the treatment of arthritis, trauma, and other medical conditions leading to joint destruction and pain, as well as methods of use. In various cases, the prosthesis does not require reaming into the intramedullary canal and does not require diaphyseal fit for any bones associated with the joint, thereby resulting in more rapid and complete recovery. In various cases, the prosthesis incorporates an anchoring stem specifically designed to be impacted into the bone and to facilitate bony ingrowth after implantation, thereby strengthening the implanted prosthesis and preventing rotation thereof.

### CROSS-REFERENCE TO RELATED APPLICATIONS

This application claims the benefit of U.S. Provisional Application No. 61/340,085, filed on Mar. 11, 2010. The disclosure of which is incorporated herein by reference in its entirety.

### FIELD

The present disclosure relates to a total or partial joint prosthesis and method, each of which enables the characteristics of a natural joint to be reproduced with good precision.

### BACKGROUND

The statements in this section merely provide background information related to the present disclosure and may not constitute prior art.

Joint arthroplasty and hemiarthroplasty are commonly used treatment methods for reconstruction of shoulder and hip injuries. Currently, as many as 23,000 Americans receive shoulder arthroplasty and approximately 120,000 Americans undergo hip arthroplasty each year. These procedures are performed to treat conditions such as osteoarthritis, rheumatoid arthritis, post-traumatic arthritis, rotator cuff tear arthropathy (a combination of severe arthritis and a massive non-reparable rotator cuff tendon tear), avascular necrosis (osteonecrosis), unsuccessful previous joint replacement surgery, and traumatic joint injuries.

As the average age of society increases and the expected quality of medical treatment increases, the occurrence of surgical procedures to repair or replace worn arthritic joints correspondingly increases. Consequently, the surgical replacement of articulating joints is becoming more common. In the traditional replacement of an articulating joint, one end of the prosthesis is placed within a bone on one side of a joint. Placing the prosthesis within the bone allows adequate mechanical stabilization between the bone and the implant. The opposite side of the prosthesis is configured to functionally replace the removed articulating end of the bone and provide a joint articulation surface.

In this traditional procedure, bone is normally prepared for the prosthesis by first resecting the bone and removing the damaged articulating end of the bone. This exposes the inside of the bone. Then, in the case of long bones, tools such as reamers, broaches and other bone tissue removal instruments are used to create a bone cavity that extends from the resection down into the intramedullary canal. Often times bone cement is then added to the cavity, creating a bone/cement mantle between the prosthesis and the bone. Sometimes the shape of the cavity is prepared to closely match the external surface of the prosthesis such that no bone cement is used.

Once the bone cavity is prepared, the prosthesis is placed into the bone cavity and is supported by the internal bone tissue or bone/cement mantle. Then, the prosthesis is positioned such that the articulating end of the implant articulates with the opposite side of the natural joint in the case of a hemiarthroplasty, or articulates with a corresponding implant replacing the opposite side of the joint in the case of an arthroplasty.

However, the traditional procedure for joint reconstruction or treatment, involving full resection of the bone and reaming of the intramedullary canal or the long stem, is highly invasive and can result in additional trauma from the surgical procedure itself that could complicate or impair the healing process. Additionally, such an invasive procedure can itself be more altering or damaging to the joint and the bones associated therewith than the injury or condition the procedure is intended to remedy.

A successful joint replacement procedure restores the biomechanical function of the joint while maintaining a secure interface with the bone, thereby allowing the loads on the joint to be distributed optimally. A closely matching fit between the prosthesis and the bone issue helps to stabilize the prosthesis and transfer the loads from the implant to the bone efficiently. Achieving such a matching fit for each patient, wherein the patients will vary greatly in size and bone structure, requires operating centers to keep in inventory an extraordinary number of single piece prostheses to provide single piece prostheses that optimally fit each size and shape of patient requiring a joint replacement surgery. Keeping such a large inventory of single piece prostheses can be burdensome and cost inefficient. To address this, often operating centers will stock only the most common prostheses shapes and sizes. However, this can result in some patients receiving prostheses that are not ideally suited for their bone anatomy.

Modular components of joint reconstruction implants have been developed as an alternative to single piece joint reconstruction prostheses to help reduce inventory and optimize fit. Such modular components are generally separate components, wherein each component comprises a particular functional portion of single piece joint prostheses, that are connectable to form a comprehensive joint prostheses. Each of these components is available in a variety of shapes and a range of sizes. The shape and size of each component that best fits a given patent's anatomy are selected and supplied to the surgeon at the time of surgery to provide the optimal combination of components. These modular components are then mated together and secured by the mechanical connections between them.

However, the use of such known modular implants is still requires the use reamers, broaches and other bone tissue removal instruments to create a bone cavity that extends from the resection down into the intramedullary canal. Hence, joint arthroplasty and hemiarthroplasty remain very invasive procedures that can result recovery complications such as a long healing process, further injury to patient, disease, pain, and arthritis.

### SUMMARY

In various cases, the present disclosure provides a fixation device for securing a bone replacement structure of an articulating joint prosthesis to a resected end of a bone of the articulating joint. In such cases, the fixation device comprises a head having a substantially flat undersurface structured to be disposed substantially flush with a resected surface of the bone when the fixation device is implanted in the resected end of the bone. Additionally, the fixation device comprises an anchoring stem extending from the head undersurface, wherein the anchoring stem includes one or more recessed flutes helically disposed along a length of the stem, and a sharpened bone piercing tip formed at a distal end thereof. The piercing tip is structured to cut and displace the tissue of the bone upon impacting the stem into the resected end of the bone.

In various other cases, the present disclosure provides an articulating joint prosthesis that comprises a bone replacement structure that is structured and operable to replace a removed end portion of a bone of an articulating joint, and a fixation device that is structured and operable to secure the bone replacement structure to a resected end of the bone. In such cases, the fixation device includes a head having a substantially flat undersurface structured to be disposed substantially flush with a resected surface of the resected end of the bone when the fixation device is implanted in the resected end of the bone, and an anchoring stem extending from the head undersurface. The anchoring stem includes one or more recessed flutes helically disposed along a length of the anchoring stem, and a sharpened bone piercing tip formed at a distal end thereof. The piercing tip is structured to cut and displace the tissue of the bone upon impacting the anchoring stem into the resected end of the bone.

In yet other cases, the present disclosure provides an articulating joint prosthesis system that comprises a bone replacement structure that is structured and operable to replace a removed end portion of a bone of an articulating joint, a fixation device that is structured and operable to secure the bone replacement structure to a resected end of the bone. In such cases, the fixation device includes a head having a substantially flat undersurface that is structured to be disposed substantially flush with a resected surface of the resected end of the bone when the fixation device is implanted in the resected end of the bone, and an anchoring stem extending from the head undersurface. The anchoring stem comprises one or more recessed flutes helically disposed along a length of the anchoring stem, and a sharpened bone piercing tip formed at a distal end thereof. The piercing tip is structured to cut and displace the tissue of the bone upon impacting the anchoring stem into the resected end of the bone. In such cases, the articulating joint prosthesis system further includes a guide pin that is structured to be disposed into the resected end of the bone at a desired location on the resected surface having a trajectory that is substantially orthogonal with the resected surface and having a proximal end portion extending away from the resected surface. The guide pin is operable to control the placement and trajectory of the anchoring stem such that the anchoring stem can be impacted into the resected end of the bone at the desired location on the resected surface and have a trajectory that is substantially orthogonal with the resected surface.

In still yet other cases, the present disclosure provides a method for attaching an articulating joint prosthesis to a resected end of a bone of the articulating joint, wherein the method comprises disposing a guide pin into a resected end of a bone of an articulating joint at a desired location on a resected surface of the resected end such that the guide pin has a trajectory that is substantially orthogonal with the resected surface and has a proximal end portion extending away from the resected surface. The method additionally comprises placing a fixation device over the proximal end portion of the guide pin such that the proximal end portion extends through a longitudinal bore of the fixation device. The fixation device additionally includes a head having a substantially flat undersurface structured to be disposed substantially flush with the resected surface when the fixation device is implanted in the resected end of the bone, and an anchoring stem extending from the head undersurface. The anchoring stem includes one or more recessed flutes helically disposed along a length of the anchoring stem and a sharpened bone piercing tip formed at a distal end thereof. The piercing tip is structured to cut and displace the tissue of the bone upon impacting the anchoring stem into the resected end of the bone.

The method further comprises impacting the anchoring stem into the resected end along the guide pin such that the anchoring stem is disposed within the resected end of the bone at the desired location on the resected surface and has a trajectory that is substantially orthogonal with the resected surface. Further yet, the method comprises attaching a bone replacement structure to the fixation device head after the anchoring stem has been impacted into the resected end of the bone. The bone replacement structure is structured and operable to replace a removed end portion of the bone.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is an illustration of an articulating joint prosthesis affixed to a resected ball end of a bone of an exemplary articulating joint, in accordance with various cases of the present disclosure.

FIG. 2 is an illustration of the articulating joint prosthesis, shown in FIG. 1, affixed to a resected socket end of a bone of an exemplary articulating joint, in accordance with various other cases of the present disclosure.

FIG. 3 is an isometric view of the articulating joint prosthesis shown in FIG. 1, in accordance with various cases of the present disclosure.

FIG. 4 is an isometric view of a fixation device of the articulating joint prosthesis shown in FIG. 1, in accordance with various cases of the present disclosure.

FIG. 5 is a side view of the fixation device shown in FIG. 4 impacted into a resected end of a bone, in accordance with various cases of the present disclosure.

FIG. 6 a side view of the fixation device shown in FIG. 4 impacted into a resected end of a bone, in accordance with various other cases of the present disclosure.

FIG. 7 is a side view of a portion of an articulating joint prosthesis system that includes the articulating joint prosthesis shown in FIGS. 1 and 2, in accordance with various cases of the present disclosure.

FIG. 8 is a side view of another portion of the articulating joint prosthesis system shown in FIG. 7 that includes the articulating joint prosthesis shown in FIGS. 1 and 2, in accordance with various cases of the present disclosure.

FIG. 9 is a side view of yet another portion of the articulating joint prosthesis system shown in FIGS. 7 and 8 that includes the articulating joint prosthesis shown in FIGS. 1 and 2, in accordance with various other cases of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

Unless otherwise defined, all technical and scientific terms used herein have the same meaning as commonly understood by one of ordinary skill in the art to which this disclosure belongs. Any use of a singular term, such as the number one (1), is intended to encompass numerical values greater than one, such as represented by the phrase “one or more.” Any use of inclusive terms such as “including” or “such as” and the like is intended to be open ended, with a meaning similar to “including, but not limited to.” All publications, patent applications, patents, and other references mentioned herein are incorporated by reference in their entirety.

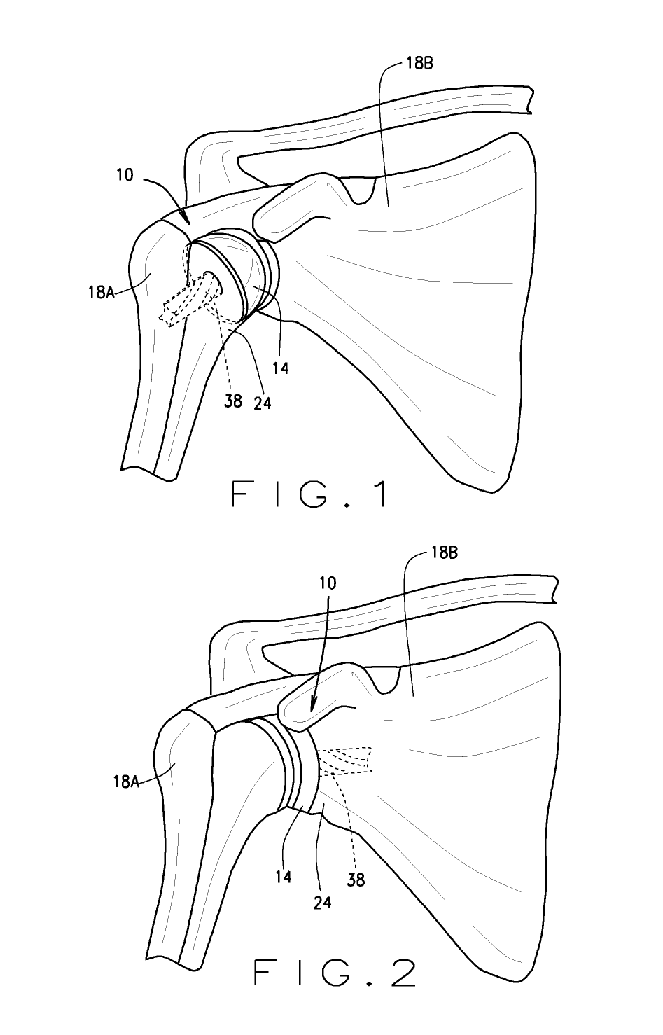

Referring to FIG. 1, the present disclosure provides an articulating joint prosthesis 10 that is a minimally invasive prosthesis for the treatment of arthritis, trauma, and other medical conditions leading to joint destruction and pain. The prosthesis 10 does not require reaming into the intramedullary canal and does not require diaphyseal fit for any bones associated with the joint, thereby resulting in more rapid and complete recovery.

It is envisioned that the prosthesis 10 is appropriate for any kind of joint treatment or technique, is suitable for replacement of either side of a joint (e.g., either the joint ball or the joint socket), and may be used alone or in combination with other implants, prosthetic and treatments. For example, the prosthesis 10 may be used in a hemiarthroplasty to treat only a single side of a joint, or may be used in combination with an opposing joint socket treatment or opposing joint ball treatment. Or, the prosthesis 10 can used in a full arthroplasty to treat both sides of a joint.

Moreover, although the prosthesis 10 is exemplarily illustrated in the various figures as being used to repair a shoulder joint by replacing a portion of the humeral head (e.g., FIG. 1) or the glenoid cavity (e.g., FIG. 2), it should be understood that the prosthesis 10, as described herein, can be used in other ball and socket joints (e.g., a hip joint) and can be used in joints that are not ball and socket joints (e.g., knee and elbow joints and the like).

Additionally, it is envisioned that the prosthesis 10 can be used to treat disease, injury, or complication of a joint of any organism having bones appropriate for functional acceptance of the prosthesis 10, including, for example, humans, domestic animals and other mammals.

Referring now to FIGS. 1, 2, 3 and 4, in various cases, the prosthesis 10 includes a bone replacement structure 14 that is structured and operable to replace a resected, i.e., removed, end portion of a bone of an articulating joint. For example, in various cases, the bone replacement structure 14 can be structured and operable to replace a resected portion of the head of a humeral or femoral bone, exemplarily illustrated and referred to herein as bone 18A. Alternatively, in various cases, the bone replacement structure 14 can be structured and operable to replace a resected portion of a glenoid cavity (i.e., shoulder socket) or an acetabulum (i.e., hip socket), exemplarily illustrated and referred to herein as bone 18B.

Referring now to FIGS. 1, 2, 3, 4 and 5, the prosthesis 10 additionally includes a fixation device 22 that is structured and operable to secure the bone replacement structure 14 to a resected end 24 of the bone 18A/18B. As described further below, the bone replacement structure 14 and the fixation device 22 are structured to be matable and connectable with each other once the fixation device 22 has been implanted, as described below, to provide the prosthesis 10.

The fixation device 22 comprises a head 26 having a substantially flat undersurface 30 that is structured to be disposed substantially flush with a resected surface 34 of the resected end 24 of the bone 18A/18B when the fixation device 22 is implanted in the resected end 24 of the bone 18A/18B, as described below. The fixation device 22 additionally includes an anchoring stem 38 that extends from the head undersurface 30. The anchoring stem 38 includes a helical flute and blade system and a sharpened bone piercing tip 46 formed at a distal end of the anchoring stem 38.

More specifically, the anchoring stem 38 includes one or more recessed flutes 42, e.g., one, two, three, four, five or more, helically disposed along a length L of the anchoring stem 38 such that the recessed flutes 42 form one or more blades 44, e.g., one, two, three, four, five or more, helically disposed along the length L of the anchoring stem 38. The flutes 42 and blades 44 twist around a central axis A of the anchoring stem 38 in a corkscrew or rotational inclined plane shape, and have any appropriate degree of rotation around the central axis A, for example, approximately 90°, 180°, 270° or 360° of rotation. Thus, when being impacted, i.e., hammered, into the bone 18A/18B, as described below, the anchoring stem 38 will rotate, or twist, the corresponding number of degrees, e.g., approximately 90°, 180°, 270° or 360°. Importantly, once the fixation device 22 is impacted into the bone 18A/18B such that the undersurface 30 of the head 26 is substantially flush against the resected surface 34, the helical disposition of the flutes 42 and blades 44 about the anchoring stem 38 and the frictional interface contact between the head undersurface 30 and resected surface 34 prevents withdrawal of the fixation device 22 from the bone 18A/18B.

That is, the helical disposition of the flutes 42 and blades 44 about the anchoring stem 38 and the frictional interface contact between the head undersurface 30 and resected surface 34 prevents the anchoring stem 38 from backing-out of or becoming loose within in the bone and further prevents fixation device 22 from being pulled out of the bone 18A/18B. Hence, upon impacting the fixation device 22 into the bone 18A/18B, the fixation device 22 is firmly disposed, seated and retained within the bone 18A/18B.

The bone piercing tip 46 of the anchoring stem 38 is structured to cut and displace the tissue of the bone 18A/18B upon impacting the anchoring stem 38 into the resected end 24 of the bone 18A/18B. More specifically, the piercing tip 46 includes a plurality of cutting edges 50 that are sharpened sufficiently to cut, separate and displace the tissue of the bone 18A/18B as the anchoring stem 38 of the fixation device 22 is impacted into the resected end 24 of the bone 18A/18B. That is, the piercing tip 46 cuts, separates and displaces the tissue of the bone 18A/18B as the anchoring stem 38 of the fixation device 22 is driven into the bone 18A/18B via a straight-line, i.e., non-rotational, impacting, or hammering, force applied to the fixation device head 26 substantially coaxially with the central axis A.

The anchoring stem 38 can generally have any desired dimension or size, but is structured and operable to firmly retain fixation device 22 within the bone 18A/18B without extending into the intramedullary canal, or shaft, of the bone 18A/18B. Hence, the anchoring stem 38 is structured to have the length L that is substantially shorter than known anchoring systems such that the anchoring stem 38 extends only into the epiphyses, or in some instances into the metaphysis, of the bone 18A/18B. Additionally, the anchoring stem 38 can be fabricated to have various lengths L and diameters D having patient-specific dimensions based on size of the patient's bone structure.

For example, in various cases, the anchoring stem 38 can be fabricated to have a diameter D of between 10 mm and 50 mm and a length L of between 20 mm and 60 mm. For instance, in various exemplary cases, a fixation device 22 having an anchoring stem 38 fabricated to have a diameter D of between 35 mm and 50 mm and a length L of between 45 mm and 60 mm can be used where a patient has a large bone structure. While in other exemplary instances, a fixation device 22 having an anchoring stem 38 fabricated to have a diameter D of between 20 mm and 35 mm and a length L of between 30 mm and 45 mm can be used where a patient has a medium sized bone structure. While in still other exemplary instances, a fixation device 22 having an anchoring stem 38 can be fabricated to have a diameter D of between 10 mm and 20 mm and a length L of between 20 mm and 30 mm can be used where a patient has a small bone structure.

With further reference to FIGS. 1, 2, 3, 4 and 5, as described above, the fixation device head 26 includes a flat undersurface 30 that is structured to be disposed substantially flush with a resected surface 34 of the resected end 24 of the bone 18A/18B when the fixation device 22 has been impacted into the resected end 24 of the bone 18A/18B. Moreover, the head 26 is structured and operable to provide a depth stop, or limiter, for fixation device 22 such that the anchoring stem 38 can only be impacted, or straight-line driven, into the bone 18A/18B a particular distance, i.e., the length L of the anchoring stem 38. Hence, as the fixation device 22 is being impacted into the bone 18A/18B the anchoring stem 38 will advance into the bone 18A/18B until the undersurface 30 of head 26 contacts the resected surface 34. At which point, the head 26 will prevent the anchoring stem 38 from advancing further into the bone 18A/18B.

In various cases, a top surface 54 of the head 26 is also substantially flat. However, it is envisioned that the top surface 54 can have any desired shape or formation suitable for mating with the bone replacement structure 14. Similarly, although the peripheral shape of the fixation device head 26 is illustrated as being substantially circular, it is envisioned that the peripheral shape of the head 26 can have any desired shape suitable for mating with the bone replacement structure 14.

Additionally, in various cases, the head 26 includes a plurality of osteotome slots 58 that extend through the head 26. The osteotome slots 58 are structured and operable to allow an osteotome to be inserted therethrough to cut the bone tissue around the anchoring stem 38 in instances where the fixation device 22 needs to be removed after being implanted, as described herein.

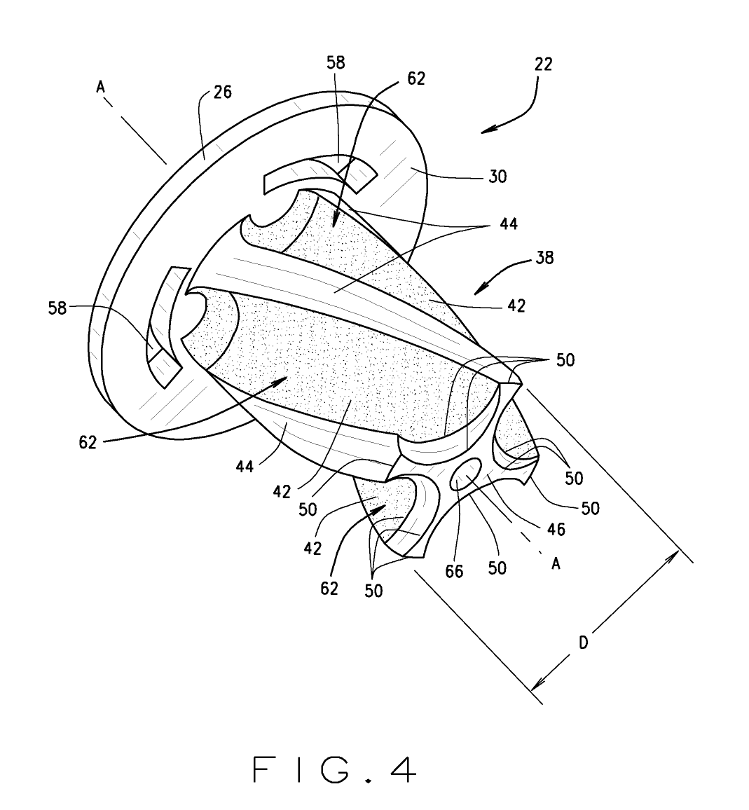

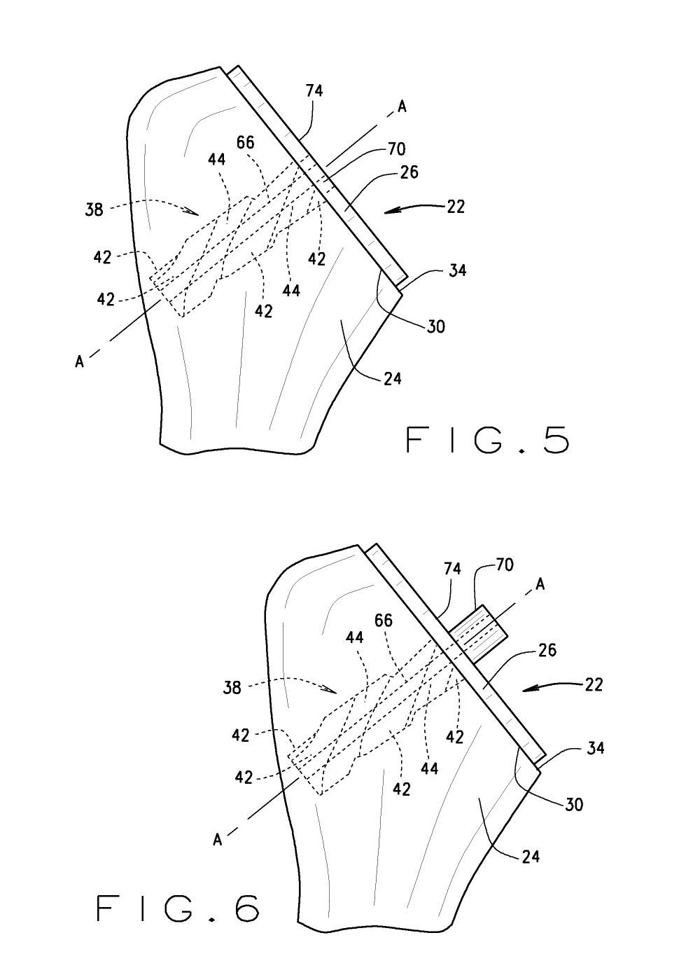

Referring now to FIG. 4, in various cases, the flutes 42 of the anchoring stem 38 include a textured surface 62 that is structured and operable to stimulate and facilitate bony ingrowth into the flutes 42 and around the blades 44 after implantation. Stimulating and facilitating the bony ingrowth, increases the permanentness of the implanted anchoring stem 38 within the bone 18A/18B enhances the strength, durability and stability of the prosthesis 10. The textured surface 62 can be any texture treatment applied to or fabricated in the flutes 42. For example, in various cases, the textured surface 62 can be integrally formed into the flutes 42 during fabrication of the fixation device 22. Alternatively, in various cases, the textured surface 62 can be applied to, i.e., adhered to, flutes 42 after the fixation device 22 is fabricated.

Additionally, the structure of the fixation device head 26 further increases the permanentness of the implanted anchoring stem 38 and further enhances strength, durability and stability of the prosthesis 10. More specifically, the contact surface area between the undersurface 30 of the fixation device head 26 and the resected surface 34 of the bone 18A/18B provides a radial lever arm for the anchoring stem 38 such that angular forces applied to prosthesis 10 are applied to and dispersed about the resected surface 34 as axial force vectors rather than being transferred to the anchoring stem 38 and applied as radial force vectors to bone 18A/18B. Furthermore, the boney ingrowth with in the anchoring stem flutes 42 in combination with frictional force existing between the undersurface 30 of the fixation device head 26 and the resected surface 34 after the fixation device 22 is impacted into the bone 18A/18B, prevent rotation of the fixation device 22 when forces are applied to the implanted prosthesis 10.

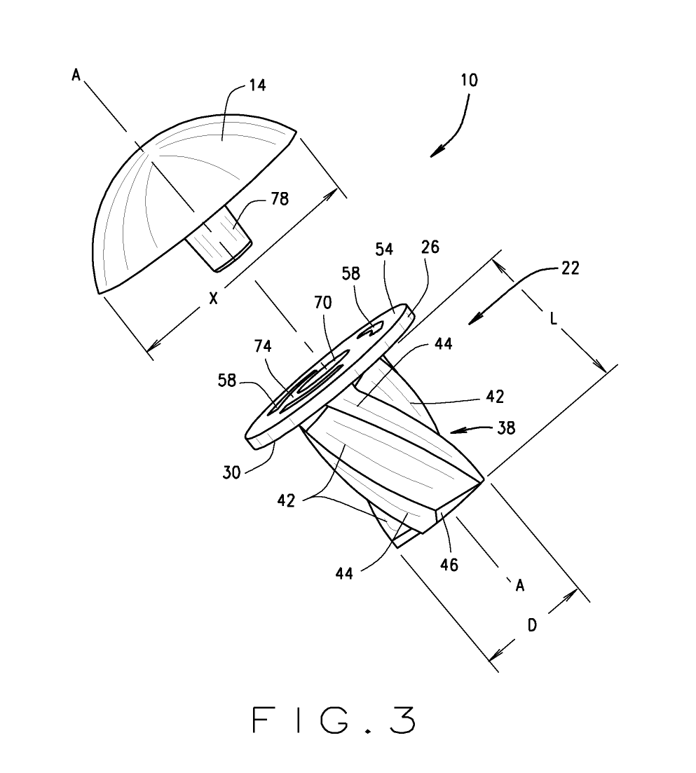

Referring now to FIGS. 3, 4, 5 and 6, in various cases the fixation device 22 includes a longitudinal bore 66 that extends along the axis A through the head 26 and the anchoring stem 38. The bore 66 is used during the process of impacting the fixation device 22 into the bone 18A/18B, as described below.

Additionally, the fixation device 22 includes a bone replacement structure coupling 70 disposed at a proximal end 74 of the fixation device 22. The bone replacement coupling 70 is structured and operable to securely couple the bone replacement structure 14 to the fixation device 22. The bone replacement structure coupling 70 can be any coupling, connector, fastening or fixation device suitable for affixing the bone replacement structure 14 to the fixation device 22 after the fixation device 22 has been implanted into the bone 18A/18B, as described herein.

For example, in various cases, as exemplarily illustrated in the FIGS. 3 and 5, the bone replacement structure coupling 70 can comprise a coupling socket axially centered within the proximal end 74 of the fixation device 22. In such cases, the coupling socket 70 is structured to receive a coupling post 78 of the bone replacement structure 14 to thereby securely couple the bone replacement structure 14 to the fixation device 22. The coupling socket 70 and the coupling post 78 can be structured to securely affix the bone replacement structure 14 to the fixation device 22 in any suitable manner. For example, in various implementations, the coupling post 78 can be structured to have Morse taper formed to be inserted into and frictionally connect within the coupling socket 70 such that the bone replacement structure 14 is securely affixed to the fixation device 22. Alternatively, the bone replacement structure 14 can be affixed to the fixation device 22 using screws, rivets, glue, nails, snaps, interlocking boss and slot connector, or any other connective device or manner suitable to securely affix the bone replacement structure 14 to the fixation device 22.

As a further example, in various cases, as exemplarily illustrated in the FIG. 6, the bone replacement structure coupling 70 can comprise a coupling boss axially centered with and extending from the proximal end 74 of the fixation device 22. In such cases, the coupling boss 70 is structured to be received within a coupling receptacle (not shown, but readily understood by one skilled in the art) of the bone replacement structure 14 to thereby securely couple the bone replacement structure 14 to the fixation device 22. The coupling boss 70 and the coupling receptacle can be structured to securely affix the bone replacement structure 14 to the fixation device 22 in any suitable manner. For example, in various implementations, the coupling boss 70 can be structured to have Morse taper formed to be inserted into and frictionally connect within the coupling receptacle such that the bone replacement structure 14 is securely affixed to the fixation device 22. Alternatively, the bone replacement structure 14 can be affixed to the fixation device 22 using screws, rivets, glue, nails, snaps, interlocking boss and slot connector, or any other connective device or manner suitable to securely affix the bone replacement structure 14 to the fixation device 22.

Referring now to FIGS. 1, 2 and 3, as described above, the bone replacement structure 14 is structured and operable to replace the resected, or removed, end portion of the bone 18A/18B of an articulating joint. For example, the bone replacement structure 14 can be structured and operable to replace the resected portion of the head of a humeral or femoral bone or to replace the resected portion of the glenoid cavity of a shoulder joint or the acetabulum of a hip joint. The bone replacement structure 14 can be fabricated of material suitable for use as a prosthetic joint implant. For example, in various cases, the bone replacement structure 14 can be fabricated from various suitable metals, e.g., stainless steel or titanium, or various suitable plastics, ceramics or other bone-like substances.

Additionally, the bone replacement structure 14 can generally have any desired shape and size suitable for imitating the natural conformation of the end of the bone 18A/18B that the prosthesis 10 is to replace. For example, in various cases, the bone replacement structure 14 can have a substantially semispherical shape to replace the resected head of a humeral or femoral bone. Or, in various other cases, the bone replacement structure 14 can have a concave shape to replace a resected glenoid cavity or acetabulum.

Furthermore, the bone replacement structure 14 can be fabricated such that various sized of bone replacement structures 14 can be mated with and connected to various sizes of fixation device 22. For example, bone replacement structures 14 having a 12 mm diameter X can be fabricated to mate with fixation devices 22 having lengths L of 40 mm, 50 mm and 60 mm, while bone replacement structures having a 10 mm diameter X can be fabricated to mate with fixation devices 22 having a lengths of 30 mm, 40 mm and 50 mm. Hence, each bone replacement structure 14, regardless of the particular size and shape of the respective bone replacement structure 14, can be mated with multiple sizes of fixation devices 22, and vice versa.

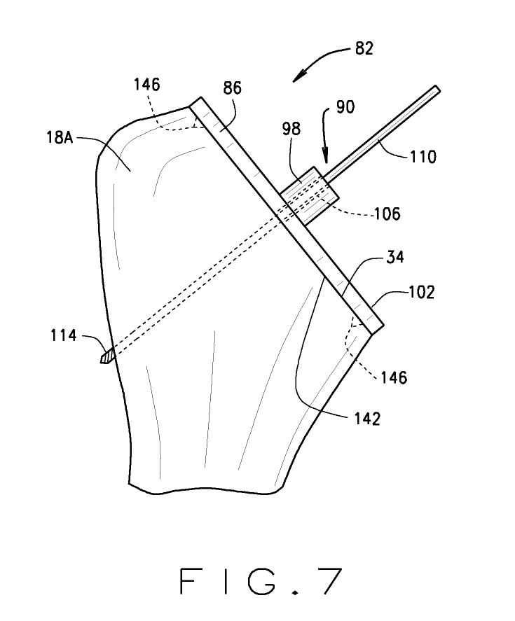

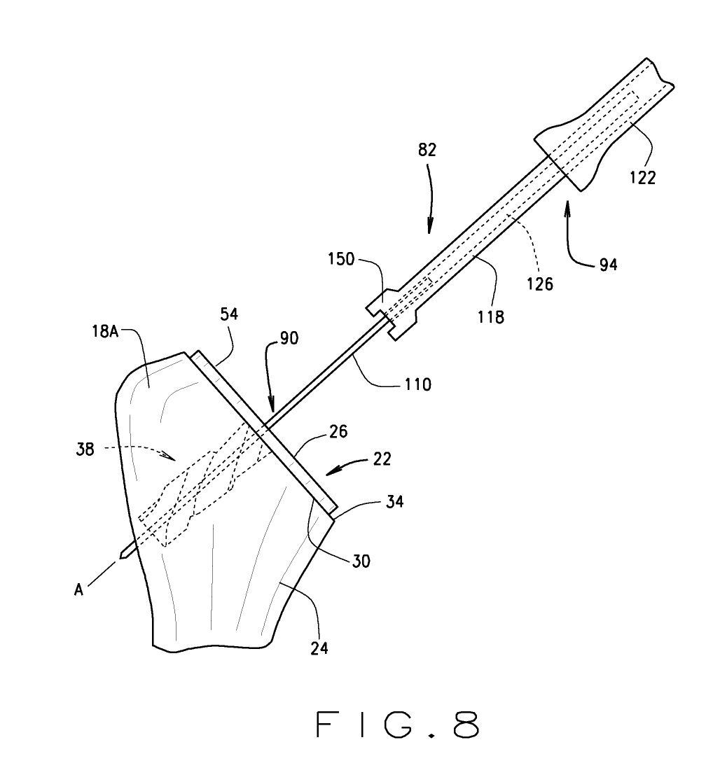

Referring now to FIGS. 7 and 8, in various cases the prosthesis 10 can be included in an articulating joint prosthesis system 82, a portion of which is shown in FIG. 7 and another portion of which is shown in FIG. 8. In various cases, the articulating joint prosthesis system 82 comprises the articulating joint prosthesis 10, as described above, a centering guide 86, a guide pin 90 and a cannulated impacting tool 94.

The centering guide 86 is structured and operable to be disposed onto the resected surface 34 in preparation of impacting the fixation device 22 in to the bone 18A/18B to control the placement and trajectory of the guide pin 90, as described below, such that the guide pin is disposed within the resected end of the bone 18A/18B at substantially the center of the resected surface 34 and has a trajectory that is substantially orthogonal with the resected surface 34. More particularly, the centering guide 86 includes a center post 98 that extends from and is substantially centered on a top surface 102 of the centering guide 86. Additionally, the centering guide 86 includes a centering aperture 106 that extends through the center post 98 and is axially centered within the centering guide 86. In use, the centering guide 86 is disposed onto the resected surface 34 such that the center aperture 106 positioned at a desired location on the resected surface 34. For example, the centering guide 86 can be disposed onto the resected surface 34 such that the center aperture 106 is positioned substantially at the center of the resected surface 34.

Generally, an advantage of the prosthesis 10 is that the anchoring stem 38 can be disposed within the center of the resected surface 34, which provides ease and consistency in implantation of the fixation device 22. However, in the cases of skeletal deformity there can be a need for offsetting of the fixation device 22. In such cases, the centering guide 86 can alternatively be disposed onto the resected surface 34 such that the center aperture 106 is positioned at a desired location substantially off-center of the resected surface 34.

The guide pin 90 is structured to be inserted through the centering aperture 106 of the centering guide 86 and disposed into the resected end of the bone 18A/18B at the location of the centering guide center aperture 106, e.g., at substantially the center of the resected surface 34, having a trajectory that is substantially orthogonal with the resected surface 34 and having a proximal end portion 110 of the guide pin 90 extending away from the resected surface 34. The guide pin 90 can be disposed into the resected end of the bone 18A/18B using any suit method of disposition. For example, in various cases, the guide pin 90 can have a drill bit formed on a distal end 114 such that the guide pin 90 is disposed within the resected end of the bone 18A/18B by drilling the guide pin 90 into the resected end of the bone 18A/18B. Alternatively, in various cases, the guide pin 90 can have a sharpened distal end 114 such that the guide pin 90 is disposed within the resected end of the bone 18A/18B by impacting the guide pin 90 into the resected end of the bone 18A/18B.

As described below, the guide pin 90 is operable to control the placement and trajectory of the anchoring stem 38 such that the anchoring stem 38 can be impacted within the resected end of the bone 18A/18B at a desire location, e.g., substantially in the center of the resected surface 34, and have a trajectory that is substantially orthogonal with the resected surface 34.

The cannulated impacting tool 94 is structured and operable to impact the fixation device 22, more particularly, the anchoring stem 38, into the resected end of the bone 18A/18B, as described below. Specifically, the fixation device 22 is impacted into the resected end of the bone 18A/18B along the guide pin 90 such that the anchoring stem 38 is disposed within the resected end of the bone at a desired location, e.g., substantially at the center of the resected surface 34, and has a trajectory that is substantially orthogonal with the resected surface 34. The impacting tool comprises a cannulated impact barrel 118 extending from an impact handle 122, wherein the impact barrel includes an internal channel 126.

In various cases, the articulating joint prosthesis system 82 can additionally include a cannulated drill bit 130 having an internal lumen 134. The cannulated drill bit 130 is structured and operable to cut a shallow starting recess 138 in the resected surface 34 to provide stability to the bone piercing tip 46 of the fixation device 22 at initiation of impacting the anchoring stem 38 of the fixation device 22 into the resected end of the bone 18A/18B, as described below.

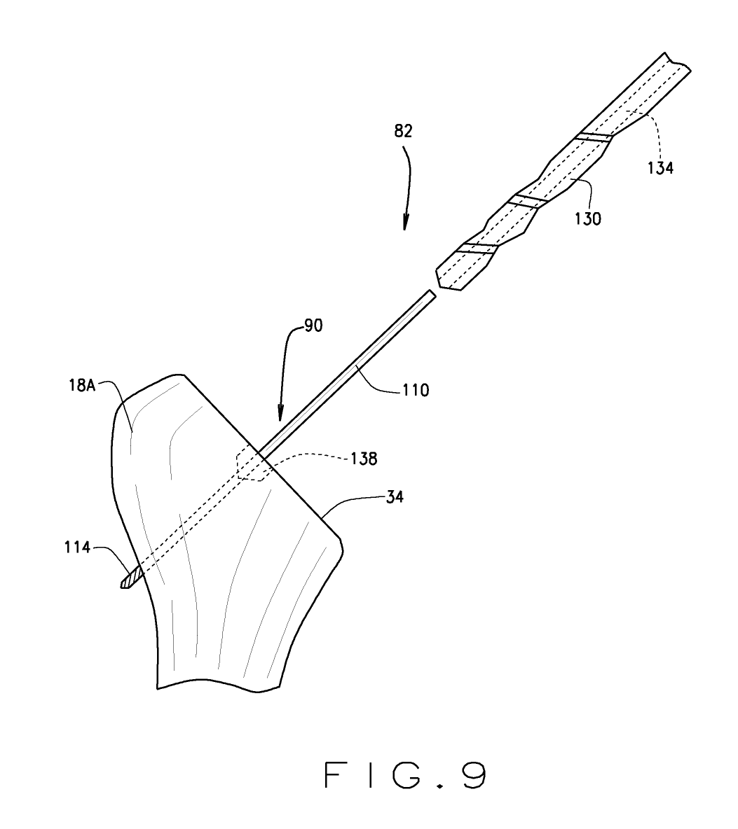

Referring now to FIGS. 1 through 9, In various cases, the articulating joint prosthesis 10 can be installed utilizing the articulating joint prostheses system 82 as follows. After the osteotomy is performed, thereby providing the resected surface the centering guide 86 is disposing on the resected surface 34. Particularly, the centering guide 86 is disposed on the resected surface 34 such that the centering aperture 106 is at a desired location on the resected surface 34, e.g., the center of the resected surface 34.

The centering guide 86 can be retained on the resected surface utilizing any suitable retention means. For example, in various cases, a bottom surface 142 of the centering guide 86 can be textured with a plurality miniature spikes or teeth 146 that can be temporarily embedded into the resected surface 34 by lightly tapping the centering guide 86 with a mallet or hammer once the centering aperture is desirably positioned. Hence, the spikes 146 would maintain the centering guide 86 in the desired location on the resected surface 34 as the guide pin 90 is disposed within the resected end of the bone 18A/18B, as described below. Alternatively, in various cases, the centering guide 86 can be temporarily screwed to the resected surface 34 using orthopedic screws (not shown) such that the centering guide 86 is maintained in the desired location on the resected surface 34 as the guide pin 90 is disposed within the resected end of the bone 18A/18B. Or, in various cases, the centering guide 86 can merely be held in place by a clinician while the guide pin 90 is disposed within the resected end of the bone 18A/18B.

After the centering guide 86 is positioned and temporarily retained at the desired location on the resected surface 34, the distal end 114 of the guide pin 90 is inserted into the centering aperture 106 of the center post 98 of the centering guide 86. Subsequently, the guide pin 90 is disposed within the resected end of the bone 18A/18B. Importantly, the centering aperture 106 of the center post 98 controls the location at which the guide pin 90 enters the resected surface and further controls the trajectory of the guide pin 90 as the guide pin 90 advances into the resected end of the bone 18A/18B. More particularly, the centering aperture 106 of the center post 98 controls the trajectory of the guide pin 90 such that guide pin 90 is disposed within the resected end of the bone 18A/18B at an angle that is substantially orthogonal to the resected surface 34.

As described above, the guide pin 90 can be advanced into the resected end of the bone 18A/18B using any suitable method, such as drilling the guide pin 90 into the resected end of the bone 18A/18B or impacting the guide pin 90 into the resected end of the bone 18A/18, any other suitable method.

Once the guide pin 90 is disposed within the resected end of the bone 18A/18B, the centering guide 86 is removed from resected surface 34.

Subsequently, the fixation device 22 is placed over the proximal end 110 of the guide pin 90 by inserting the proximal end 110 into the longitudinal bore 66 of the fixation device 22. The fixation device 22 is then slid down the guide pin 90 until the bone piercing tip 46 of the anchoring stem 38 contacts the resected surface 34.

In various cases, prior to placing the fixation device 22 over the proximal end 110 of the guide pin 90, the cannulated drill bit 130 (attached to a drill) is placed over the proximal end 110 by inserting the proximal end 110 into the internal lumen 134 of the drill bit 130. The cannulated drill bit 130 can then be rotated (via, the drill) to cut the shallow starting recess 138. As described above, in such cases, the shallow starting recess 138 is created to provide stability for the piercing tip 46 of the fixation device 22 at initiation of impacting the anchoring stem 38 into the resected end of the bone 18A/18B. Hence, the shallow starting recess 138 only extends into the resected end of the bone 18A/18B a minimal distance, e.g., approximately 1 mm to 5 mm. In such cases, once the starting recess 138 is provided, the fixation device 22 is placed over the proximal end 110 of the guide pin 90 such that the bone piercing tip 46 of the anchoring stem 38 contacts the resected surface 34 within the shallow starting recess 138.

Alternatively, in instances where the bone 18A/18B is exceptionally hard, the cannulated drill bit 130 can be utilized to drill a deeper recess 138 that can have any desirable depth. For instance, the recess 138 can be drilled to have a depth equal to ½ or ¾ the length L of the anchoring stem 38, or in some instances have a depth that is equal to or greater than the length L of the anchoring stem 38, thereby providing easier impaction of the fixation device 22 into the bone 18A/18B.

After the fixation device 22 is placed over the proximal end 110 of the guide pin 90 and the bone piercing tip 46 is in contact with the resected surface 34 (with or without the shallow starting recess 138), the cannulated impact barrel 118 of the impact tool 94 is placed over the proximal end 110 of the guide pin 90 by inserting the proximal end 110 into the internal channel 126 of the impact barrel 118. The impact barrel 118 is advanced along the guide pin proximal end 110 until a distal end 150 of the impact barrel 118 contacts the top surface 54 of the fixation device head 26. Subsequently, an impacting force is repeatedly applied to the impact tool handle 122, via a mallet or hammer. As one skilled in the art will readily understand, the impacting force applied to the handle 122 is translated through the handle 122 and impact barrel 118 such that the impacting force is applied to the fixation device head 26, and more particularly to the anchoring stem 38. Hence, the force of each impact causes the cutting edges 50 of the anchoring stem piercing tip 46 to cut, separate and displace the soft cancellous bone tissue of the resected end of the bone 18A/18B.

Accordingly, with each impact the anchoring stem 38 is advanced into the resected end of the bone 18A/18B until the undersurface 30 of the fixation device head 26 contacts the resected surface 34. At which point, the anchoring stem 38 is prevented from advancing further. Importantly, the anchoring stem 38 is advanced into the resected end of the bone 18A/18B along the guide pin 90 such that the anchoring stem 90 is disposed within the resected end of the bone 18A/18B at an angle that is substantially orthogonal to the resected surface 34.

After the fixation device 22 has been impacted into the resected end of the bone 18A/18B, the guide pin 90 is removed, i.e., withdrawn though the longitudinal bore 66 of the fixation device 22. Thereafter, the bone replacement structure 14 is securely coupled, or affixed, to the head 26 of the fixation device utilizing the bone replacement structure coupling 70, as described above. For example, in various cases wherein the bone replacement structure comprises a coupling socket 70, the coupling post 74 of the bone replacement structure 14 is secured within the coupling socket 70, e.g., via Morse taper of the coupling post 74. As also described above, the bone replacement structure 14 can be structured to replicate and replace a portion of either a joint ball or a joint socket, e.g., a portion of a humeral or femoral head 18A, or a portion of a glenoid cavity or acetabulum 18B.

In summary, the prosthesis 10, as described herein, comprises the fixation device 22 that is includes the anchoring stem 38 that is substantially shorter than known, anchoring systems. Particularly, the anchoring stem 38 of the present disclosure extends only into the epiphysis, or in some cases into the metaphysis, of the resected end of the bone 18A/18B once implanted. Moreover, as described herein, implantation of the prosthesis 10 of the present disclosure does not require any reaming of bone, because the anchoring stem 38 of the fixation device 22 includes the bone piercing tip 46 that is structured to cut, separate and displace the soft cancellous bone as it is impacted, or straight-line driven, into the resected end of the bone 18A/18B.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. A fixation device for securing a bone replacement structure of an articulating joint prosthesis to a resected end of a bone of the articulating joint, said fixation device comprising:
a head having a substantially flat undersurface structured to be disposed substantially flush with a resected surface of the bone when the fixation device is implanted in the resected end of the bone; and
an anchoring stem extending from the head undersurface, the anchoring stem comprising:
one or more recessed flutes helically disposed along a length of the anchoring stem; and
a sharpened bone piercing tip formed at a distal end thereof, the piercing tip structured to cut and displace the tissue of the bone upon impacting the anchoring stem into the resected end of the bone.

2. The fixation device of claim 1 further comprising a longitudinal bore extending through the fixation device along a center axis of the fixation device.

3. The fixation device of claim 1 further comprising a bone replacement structure coupling disposed at a proximal end of the fixation device and structured and operable to securely couple the bone replacement structure to the fixation device.

4. The fixation device of claim 3, wherein the bone replacement structure coupling comprises a coupling socket axially centered within the proximal end of the fixation device, the coupling socket structured to receive a coupling post of the bone replacement structure to thereby securely couple the bone replacement structure to the fixation device.

5. The fixation device of claim 3, wherein the bone replacement structure coupling comprises a coupling boss axially centered with and extending from the proximal end of the fixation device, the coupling boss structured to be received within a coupling receptacle of the bone replacement structure to thereby securely couple the bone replacement structure to the fixation device.

6. The fixation device of claim 1, wherein at least one of the one or more helically disposed flutes includes a textured surface structured and operable to promote bone growth within each respective helically disposed flute.

7. The fixation device of claim 1, wherein the head is structured to provide a depth stop operable to control the distance the anchoring stem can be impacted into the resected end of the bone.

8. The fixation device of claim 1, wherein the head comprises a plurality of osteotome slots structured to allow insertion of an osteotome to remove the fixation device from the resected end of the bone after the fixation device has been implanted.

9. An articulating joint prosthesis, said prosthesis comprising:
a bone replacement structure that is structured and operable to replace a removed end portion of a bone of an articulating joint; and
a fixation device structured and operable to secure the bone replacement structure to a resected end of the bone, said fixation device comprising:
a head having a substantially flat undersurface structured to be disposed substantially flush with a resected surface of the resected end of the bone when the fixation device is implanted in the resected end of the bone; and
an anchoring stem extending from the head undersurface, the anchoring stem comprising:
one or more recessed flutes helically disposed along a length of the anchoring stem; and
a sharpened bone piercing tip formed at a distal end thereof, the piercing tip structured to cut and displace the tissue of the bone upon impacting the anchoring stem into the resected end of the bone.

10. The prosthesis of claim 9 further comprising a longitudinal bore extending through the fixation device along a center axis of the fixation device.

11. The prosthesis of claim 9, wherein the fixation device further comprises a bone replacement structure coupling disposed at a proximal end of the fixation device and structured and operable to securely couple the bone replacement structure to the fixation device.

12. The prosthesis of claim 11, wherein the bone replacement structure coupling comprises a coupling socket axially centered within the proximal end of the fixation device, the coupling socket structured to receive a coupling post of the bone replacement structure to thereby securely couple the bone replacement structure to the fixation device.

13. The prosthesis of claim 11, wherein the bone replacement structure coupling comprises a coupling boss axially centered with and extending from the proximal end of the fixation device, the coupling boss structured to be received within a coupling receptacle of the bone replacement structure to thereby securely couple the bone replacement structure to the fixation device.

14. The prosthesis of claim 9, wherein at least one of the one or more helically disposed flutes includes a textured surface structured and operable to promote bone growth within each respective helically disposed flute.

15. The prosthesis of claim 9, wherein the head of the fixation device comprises a plurality of osteotome slots structured to allow insertion of an osteotome to remove the fixation device from the resected end of the bone after the fixation device has been implanted.

16. The prosthesis of claim 9, wherein the bone replacement structure comprises a substantially semispherical bone head prosthetic.

17. An articulating joint prosthesis system, said system comprising:
a bone replacement structure that is structured and operable to replace a removed end portion of a bone of an articulating joint;
a fixation device structured and operable to secure the bone replacement structure to a resected end of the bone, said fixation device comprising:
a head having a substantially flat undersurface structured to be disposed substantially flush with a resected surface of the resected end of the bone when the fixation device is implanted in the resected end of the bone; and
an anchoring stem extending from the head undersurface, the anchoring stem comprising:
one or more recessed flutes helically disposed along a length of the anchoring stem; and
a sharpened bone piercing tip formed at a distal end thereof, the piercing tip structured to cut and displace the tissue of the bone upon impacting the anchoring stem into the resected end of the bone; and
a guide pin structured to be disposed into the resected end of the bone at a desired location on the resected surface having a trajectory that is substantially orthogonal with the resected surface and having a proximal end portion extending away from the resected surface, the guide pin operable to control the placement and trajectory of the anchoring stem such that the anchoring stem can be impacted into the resected end of the bone at the desired location on the resected surface and have a trajectory that is substantially orthogonal with the resected surface.

18. The prosthesis system of claim 17 further including a centering guide structured and operable to control the placement and trajectory of the guide pin such that the guide pin is disposed within the resected end of the bone at the desired location on the resected surface and has a trajectory that is substantially orthogonal with the resected surface.

19. The prosthesis system of claim 18, wherein the anchoring stem further comprising a longitudinal bore extending through the fixation device along a center axis of the fixation device such that the proximal end portion of the guide pin can be inserted through the bore of anchoring stem.

20. The prosthesis system of claim 19 further including an impacting tool structured and operable to impact the anchoring stem into the resected end of the bone along the guide pin such that the anchoring stem is disposed within the resected end of the bone at the desired location on the resected surface and has a trajectory that is substantially orthogonal with the resected surface.

21. The prosthesis system of claim 17, wherein the fixation device further comprises a bone replacement structure coupling disposed at a proximal end of the fixation device and structured and operable to securely couple the bone replacement structure to the fixation device.

22. The prosthesis system of claim 17, wherein at least one of the one or more helically disposed flutes includes a textured surface structured and operable to promote bone growth within each respective helically shaped flute.

23. The prosthesis system of claim 17, wherein the head of the fixation device comprises a plurality of osteotome slots structured to allow insertion of an osteotome to remove the fixation device from the resected end of the bone after the fixation device has been implanted.

24. The prosthesis system of claim 17, wherein the bone replacement structure comprises a substantially semispherical bone head prosthetic.

25. A method for attaching an articulating joint prosthesis to a resected end of a bone of the articulating joint, said method comprising:
disposing a guide pin into a resected end of a bone of an articulating joint at a desired location on a resected surface of the resected end such that the guide pin has a trajectory that is substantially orthogonal with the resected surface and has a proximal end portion extending away from the resected surface;
placing a fixation device over the proximal end portion of the guide pin such that the proximal end portion extends through a longitudinal bore of the fixation device, the fixation device further comprising:
a head having a substantially flat undersurface structured to be disposed substantially flush with the resected surface when the fixation device is implanted in the resected end of the bone; and
an anchoring stem extending from the head undersurface, the anchoring stem comprising:
one or more recessed flutes helically disposed along a length of the anchoring stem; and
a sharpened bone piercing tip formed at a distal end thereof, the piercing tip structured to cut and displace the tissue of the bone upon impacting the anchoring stem into the resected end of the bone;
impacting the anchoring stem into the resected end along the guide pin such that the anchoring stem is disposed within the resected end of the bone at the desired location on the resected surface and has a trajectory that is substantially orthogonal with the resected surface; and
attaching a bone replacement structure to the fixation device head after the anchoring stem has been impacted into the resected end of the bone, the bone replacement structure being structured and operable to replace a removed end portion of the bone.

26. The method of claim 25, wherein disposing the guide pin into the resected end of a bone comprises:
positioning a centering guide onto the resected surface such that a cannulated center post of the centering guide is position at the desired location on the resected surface;
inserting a distal end of the guide pin into an axial duct extending through the center post; and
disposing the guide pin into the resected end of the bone utilizing the center post to control the placement and trajectory of the guide pin such that the guide pin is disposed within the resected end of the bone at the desired location on the resected surface having a trajectory that is substantially orthogonal with the resected surface and has the proximal end extending away from the resected surface.

27. The method of claim 26, wherein placing the fixation device of the proximal end of the guide pin comprises:
placing a cannulated drill bit over the proximal end of the guide pin such that the proximal end is positioned within a longitudinal center lumen of the drill bit;
utilizing the cannulated drill bit to cut a starting recess in the resected surface around the guide pin, the starting recess operable to provide stability to the piercing tip of the fixation device anchoring stem when the fixation device is placed over the proximal end of the guide pin such that the piercing tip is in contact with starting recess; and
placing the fixation device over the proximal end of the guide pin such that the piercing tip is in contact with starting recess.

28. The method of claim 26, wherein impacting the anchoring stem into the resected end comprises:
placing a cannulated drive shaft of an impact tool over the proximal end of the guide pin such the proximal end is positioned within a longitudinal center cavity of the drive shaft and a distal end of the drive shaft is in contact with a top surface of the fixation device head;
impacting the impact tool such that the drive shaft impacts the fixation device anchoring stem into the resected end of the bone along the guide pin so that the anchoring stem is disposed within the resected end of the bone at the desired location on the resected surface and has a trajectory that is substantially orthogonal with the resected surface; and
removing the guide pin.
